(19) United States
(12) Patent Application Publication
(10) Pub. No.: US 2024/0016438 A1
(43) Pub. Date: Jan. 18, 2024

(54) Segmenting audiences using brain type information

(71) Applicant: Brainvivo Ltd., Tel Aviv (IL)

(72) Inventors: Assaf Horowitz, Tel Aviv (IL); Yaniv Assaf, Tel Aviv (IL); Sharon Rousso, Tel Aviv (IL)

(21) Appl. No.: 17/863,435

(22) Filed: Jul. 13, 2022

(51) Int. Cl. A61B 5/00; A61B 5/16; A61B 5/11

(52) U.S. Cl. CPC A61B 5/4064; A61B 5/163; A61B 5/165; A61B 5/11

(57) ABSTRACT

A method for content delivery includes dividing a reference group of human subjects into multiple segments according to one or more segmentation criteria. Subjective responses of the human subjects to a reference set of data items are collected, and neurophysiological responses of the human subjects to the data items in the reference set are measured. The human subjects are classified into multiple brain types according to the measured neurophysiological responses. Based on the collected subjective responses, a mapping is defined between the segmentation criteria and the brain types and is applied in predicting a brain type of a human subject outside the reference group. A content offering is selected for presentation to the human subject responsively to the predicted brain type.

### FIELD OF THE INVENTION

The present invention relates generally to personalized content selection and delivery, and particularly to methods, systems, and software that use brain response models in selecting content offerings.

### BACKGROUND

Personalized content delivery enables organizations to deliver content to users that is tailored to meet the specific needs and interest of each user. The term “content,” in the context of the present description and in the claims, is used broadly to refer to all kinds of information that can be delivered over media channels. Thus, content may include (by way of example and not limitation) news, entertainment, sales offerings, calls to action, and invitations.

Organizations typically personalize content on the basis of users' responses to earlier content offerings (for example, by collecting user clicks on a Web site), as well as segmentation of demographic and psychographic information regarding the users. Demographics include statistical data, such as age, gender, income, and ethnicity. Psychographics include information about a person's attitudes, beliefs, values, fears, emotional state, and other psychological criteria. The term “segmentation,” in the context of the present description and in the claims, refers to classification of people according to demographic and/or psychographic criteria.

Brain activity mapping has been performed in various ways and for various purposes. For example, U.S. Patent Application Publication 2020/0170524, whose disclosure is incorporated herein by reference, describes a computer-implemented method, which includes supplying stimuli to an organism, measuring brain activity of the organism responsive to the stimuli and producing a brain feature activity map characterizing the brain activity. The operations of supplying, measuring, and producing are repeated for different stimuli to form a brain feature activity map database. New stimuli are received. New stimuli features are mapped to a projected brain activity map. The projected brain activity map is compared to the brain feature activity map database to identify similarities and dissimilarities between the projected brain activity map and entries in the brain feature activity map database to designate a match. The new stimuli are characterized based upon the match.

As another example, U.S. Patent Application Publication 2021/0241065, whose disclosure is incorporated herein by reference, describes a content classification method, which includes receiving a set of content items from categories of a specific discipline, and extracting respective features from each content item. A labeling of the content items of the specific discipline is received, performed by human viewers, the labeling indicating a respective category assigned to the content item by the human viewers. A general-content brain-response model is uploaded, the model estimated using measurements of brains of humans presented with a general-content database defined using a set of features and includes a mapping between the set of features and a set of extracted brain activities. The model is applied to the extracted features, to calculate, using the labeling, a set of brain-responses for the specific discipline. Given a new content item associated with the discipline, a category of the discipline best matching the new content item is estimated, based on the model and the discipline-specific brain responses.

### SUMMARY

Embodiments of the present invention that are described hereinbelow provide improved methods, systems, and software for personalized content selection.

There is therefore provided, in accordance with an embodiment of the invention, a method for content delivery, which includes dividing a reference group of human subjects into multiple segments according to one or more segmentation criteria. Subjective responses of the human subjects to a reference set of data items are collected, and neurophysiological responses of the human subjects to the data items in the reference set are measured. The human subjects are classified into multiple brain types according to the measured neurophysiological responses. Based on the collected subjective responses, a mapping is defined between the segmentation criteria and the brain types. The mapping is applied in predicting a brain type of a human subject outside the reference group. A content offering is selected for presentation to the human subject responsively to the predicted brain type.

In one embodiment, applying the mapping includes predicting the brain type based on a behavior of the human subject. Additionally or alternatively, applying the mapping includes predicting the brain type based on an interaction of the human subject with an item of content.

In one embodiment, the segmentation criteria include demographic criteria. Additionally or alternatively, the segmentation criteria include psychographic criteria, which may include one or more measures of mental health of the human subjects.

In some embodiments, selecting the content offering includes presenting a media item to the human subject. Additionally or alternatively, selecting the content offering includes modifying a physical property of an output presented to the human subject. Further additionally or alternatively, selecting the content offering includes presenting a proposal to the human subject to make an acquaintance with another person or presenting a proposal to the human subject to join an organization.

In some embodiments, measuring the neurophysiological responses includes collecting respective signals from one or more region of respective brains of the human subjects, and classifying the human subjects includes clustering the human subjects according to the respective signals. In one embodiment, collecting the respective signals includes receiving magnetic resonance imaging (MRI) data.

Additionally or alternatively, measuring the neurophysiological responses includes sensing vital signs of the human subjects, sensing gestures made by the human subjects, and/or measuring a dilation of pupils of the eyes of the human subjects.

In some embodiments, defining the mapping includes extracting features from the data items. A first classification of the neurophysiological responses of the human subjects to each of the extracted features is defined according to the brain types of the human subjects. A second classification of the subjective responses of the human subjects to each of the extracted features is defined according to the segments to which the human subjects belong. The first and second classifications are applied in mapping between the segmentation criteria and the brain types.

In one embodiment, the data items include images, and the extracted features are selected from among spatial and spectral characteristics of the images. In another embodiment, the data items include audio items, and the extracted features are selected from among spectrograms and spectral characteristics of the audio waves. In a further embodiment, the data items include odors, and the extracted features are selected from among spectroscopic data and chemical characteristics of the odors. In yet another embodiment, the data items include flavors, and the extracted features are selected from among spectroscopic data and chemical characteristics of the flavors. In still another embodiment, the data items include tactile stimuli, and the extracted features are selected from among vibrograms and spectral characteristics of the tactile stimuli.

In a disclosed embodiment, defining the first classification includes measuring a brain activity of the human subjects from one or more brain regions, and classifying each of the features according to the measured brain activity.

Additionally or alternatively, defining the second classification includes computing an arousal score with respect to each of the data items based on the subjective responses, and classifying each of the features according to the arousal score.

In a disclosed embodiment, predicting the brain type includes presenting a data item to the human subject, receiving a response of the human subject to the presented data item, and predicting the brain type based on the received response. Additionally or alternatively, predicting the brain type includes receiving segmentation data with respect to the human subject, and predicting the brain type based on the segmentation data.

There is also provided, in accordance with an embodiment of the invention, apparatus for content delivery, including a memory, configured to receive and store subjective responses of a reference group of human subjects to a reference set of data items and to receive and store neurophysiological responses of the human subjects to the data items in the reference set. A processor is configuring to divide the reference group of human subjects into multiple segments according to one or more segmentation criteria, to classify the human subjects into multiple brain types according to the stored neurophysiological responses, to define, based on the stored subjective responses, a mapping between the segmentation criteria and the brain types, to apply the mapping in predicting a brain type of a human subject outside the reference group, and to select a content offering for presentation to the human subject responsively to the predicted brain type.

There is additionally provided, in accordance with an embodiment of the invention, a computer software product, including a tangible, non-transitory computer-readable medium in which program instructions are stored, which instructions, when read by a computer, cause the computer to receive and store subjective responses of a reference group of human subjects to a reference set of data items and to receive and store neurophysiological responses of the human subjects to the data items in the reference set. The instructions cause the computer to divide the reference group of human subjects into multiple segments according to one or more segmentation criteria, to classify the human subjects into multiple brain types according to the stored neurophysiological responses, to define, based on the stored subjective responses, a mapping between the segmentation criteria and the brain types, to apply the mapping in predicting a brain type of a human subject outside the reference group, and to select a content offering for presentation to the human subject responsively to the predicted brain type.

The present invention will be more fully understood from the following detailed description of the embodiments thereof, taken together with the drawings in which:

### BRIEF DESCRIPTION OF THE DRAWINGS

FIG. 1 is block diagram that schematically illustrates a system for audience segmentation, in accordance with an embodiment of the invention;

FIG. 2 is a flow chart that schematically illustrates a method for mapping between segmentation of an audience and brain types, in accordance with an embodiment of the invention;

FIG. 3 is a plot that schematically illustrates emotional scoring of a set of images, in accordance with an embodiment of the invention;

FIG. 4A is a schematic representation of an image used in emotional scoring, in accordance with an embodiment of the invention;

FIGS. 4B and 4C are plots that schematically illustrate aggregated emotional scoring of the image in FIG. 4A by two groups of subjects, in accordance with an embodiment of the invention;

FIGS. 4D and 4E are statistical plots showing the distribution of emotional arousal scores among the two groups of subjects of FIGS. 4B and 4C, respectively, in accordance with an embodiment of the invention;

FIG. 5 is a plot that schematically illustrates differential arousal values computed between the two groups of FIGS. 4D and 4E for a set of images, in accordance with an embodiment of the invention;

FIG. 6 is a plot that schematically illustrates brain activity responses to a set of images, in accordance with an embodiment of the invention;

FIG. 7 is a plot that schematically illustrates a method of classifying brain types according to brain activity response, in accordance with an embodiment of the invention;

FIG. 8 is a flow chart that schematically illustrates a method for content selection based on mapping of brain types to segmentation of users of the content, in accordance with an embodiment of the invention; and

FIG. 9 is a flow chart that schematically illustrates a method for content selection base on mapping of user segmentation to brain types, in accordance with an embodiment of the invention.

### DETAILED DESCRIPTION OF EMBODIMENTS

### Overview

Targeted content delivery is a key component in the strategies and business models of most major organizations that do business over the Internet. For this purpose, organizations amass huge amounts of information concerning the interests, preferences, demographics, and psychographics of their customers, as well as potential customers. Based on this information, organizations segment their customer base and choose content offerings based on this segmentation.

In many cases, however, organizations still do not have sufficient information to select optimal content offerings for all users. For example, many users are reluctant to provide demographic information, and organizations may be prevented from soliciting or storing such information by privacy concerns and other legal considerations. Psychographic information can be even harder to collect and often varies over both short and long time scales, for example due to changes in users' views and emotional state. When a new or anonymous user approaches an organization, for example by connecting to a Web site or service offered by the organization, the organization may have little or no demographic and psychographic information at all with respect to the user.

Embodiments of the present invention that are described herein address this problem by predicting the brain type of the user, and selecting a content offering for presentation to the user based on the predicted brain type. The term “brain type,” in the context of the present description and in the claims, refers to a categorization of neurophysiological responses of human subjects to particular types of stimuli. People can be grouped empirically into brain types by measuring and clustering their responses to such stimuli.

In other words, people who respond similarly to a certain set of features of the stimuli are considered to belong to the same brain type, and conversely, people belonging to the same brain type will generally exhibit similar responses to features of the stimuli. In the embodiments that are described below, the stimuli have the form of data items, such as images, and the brain types are defined by presenting the data items to a reference group of human subjects while the neurophysiological responses of the subjects are measured. Alternatively or additionally, other sorts of stimuli may be used in defining the brain types. Brain type can include not only static features of the neurophysiological response, but also dynamic features, resulting from changes in a subject's emotional and cognitive state.

In the disclosed embodiments, a computer creates a mapping between brain types and segmentation criteria over a reference group of human subjects. The reference group is divided into multiple segments according to one or more segmentation criteria, which may include both demographic and psychographic criteria, based on information supplied by or about the subjects in the reference group. The computer collects two sets of information from the subjects in the reference group:
1. Subjective responses to a reference set of data items. These subjective responses typically comprise the subjects' answers to questions regarding the emotional impact of the data items, for example indicating the types and degrees of emotional arousal that the subjects felt.
2. Neurophysiological responses of the subjects to the data items in the reference set, for example in the form of signals, such as magnetic resonance imaging (MRI) data, and particularly function MRI (fMRI) data, originated from the subjects' brains while viewing the data items, or other direct or indirect measures of brain response, such as pupil dilation.

As explained above, the computer classifies the subjects in the reference group into multiple brain types according to their measured neurophysiological responses. The computer then applies the collected subjective responses to the data items of the subjects within each segment (as defined by the applicable segmentation criteria) in creating a mapping between the segmentation criteria and the brain types.

This mapping is applied in predicting the brain type of new subjects outside the reference group, such as new users who connect to a server offering content of a certain type. For example, the server may present a data item to a new user and predict the user's brain type based on a response received from the user to the presented data item, without acquiring any neurophysiological data from the new user. As another example, the server may receive segmentation data with respect to the new user, such as demographic and/or psychographic information input by the user, and predict the brain type based on the segmentation data. The server uses the predicted brain type in selecting content that is optimized for this brain type and offers the selected content to the user. The content offering can take various forms, such as a media item (including promotional media items with respect to a certain product or service), a proposal to the user to make an acquaintance with another person (as in a matchmaking service), or a proposal to the human subject to join an organization, including proposals such as job offers. As yet another example, the server may use the brain type in adjust digital content offered to the user on the metaverse, including adjusting the user's avatar and its surrounding.

Thus, by predicting and applying user brain types, organizations can better focus and optimize the content that they offer to users, even on the basis of limited input information from the users. For example, based on the user's interaction with a Web site, a server may predict the user's brain type and customize the set of products and/or the visual, textual, and audio characteristics of promotional content presented to the user according to the brain type. The content presented to the user may include items that appear to be unrelated to the user's activity on the Web site but nonetheless match the predicted brain type. Alternatively or additionally, a physical component of an apparatus may be changed based on the user's brain type, as derived from the user's behavior or interaction with the apparatus. For example, certain properties of the outputs of the apparatus, such as visual, odor, flavor, tactile, textual, and/or audio characteristics, may be customized accordingly. On the basis of the present techniques, user engagement and commitment can be enhanced in ways that are not evident from simple analysis of the user's interaction with the content provider.

### System Description

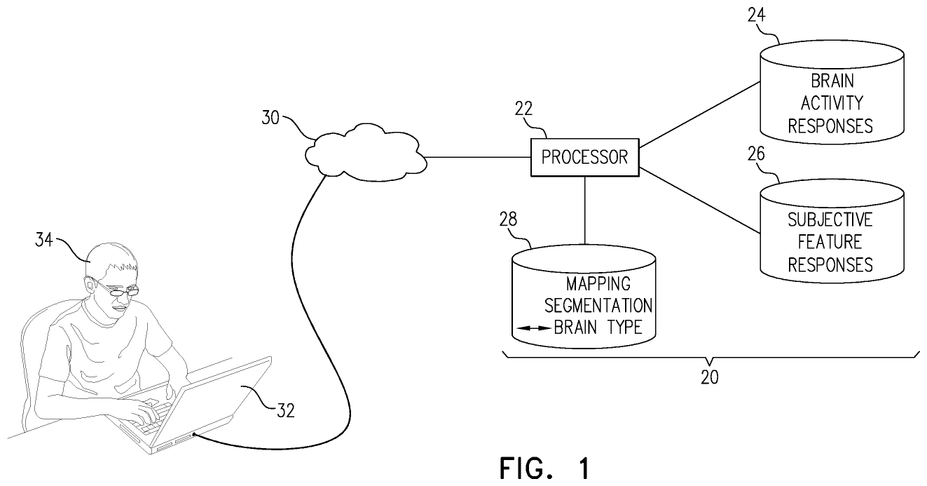

FIG. 1 is block diagram that schematically illustrates a system 20 for audience segmentation, in accordance with an embodiment of the invention. System 20 in the pictured embodiment is implemented as a server, comprising a programmable processor 22, such as central processing unit (CPU), and a memory, which stores information regarding a reference group of human subjects, for example in the form of a brain activity response database 24 and a subjective feature response database 26. Brain activity response database 24 contains data, such as fMRI data, regarding the neurophysiological responses of the subjects to the data items in a reference set, such as a set of images. Subjective feature response database 26 contains data with respect to the subjective responses of the subjects in the reference group to the data items in the same reference set.

Processor 22 also receives segmentation data with regard to the reference group of subjects, including demographic data and/or psychographic data. Based on the segmentation data, processor 22 divides the subjects in the reference group into multiple segments according to one or more segmentation criteria. The processor also classifies the subjects into multiple brain types according to the neurophysiological responses stored in database 24. Based on the stored subjective responses in database 26, processor 22 defines and stores a mapping 28 between the segmentation criteria and the brain types.

In the pictured scenario, a new subject, such as a user 34 of a computer 32, connects to system 20 via a network 30, such as the Internet. Based on inputs received from user 34, processor 22 applies mapping 28 in predicting the brain type of user 34. Processor 22 may then select content offerings for presentation to user 34 based on the predicted brain type. For example, processor 22 may use the predicted brain type in offering a media item for presentation on computer 32, such as a video clip, images, text, or offerings of products and services.

Alternatively or additionally, processor 22 may offer content of other types. For example, assuming system 20 to be connected with an appropriate sort of Web site, such as a dating site or social network, processor 22 may present a proposal to user 34 to make the acquaintance of another person, such as a possible romantic partner whose brain type is considered to be compatible with the predicted brain type of user 34. As another example, assuming system 20 to be associated with an appropriate business enterprise or other recruiting organization, processor 22 may apply the predicted brain type of user 34 in deciding whether and how to present a proposal to the user to join the organization. Other uses of the predicted brain type in interaction with human subjects will be apparent to those skilled in the art and are considered to be within the scope of the present invention.

As yet another example, the psychographic information collected by system 20 with respect to the reference group of human subjects may include measures of mental health conditions of the subjects. In this case, processor 22 may process the information in databases 24 and 26 so that mapping 28 reflects the correspondence between brain types and mental health conditions, including both cognitive and emotional conditions, as well as psychiatric pathologies. Based on the responses of user 34, processor 22 can provide feedback regarding the user's mental health, for example, cognitive improvement or decline, rehabilitation procedures, emotional stability, resilience, mental state, and/or possible toxic effects.

Processor 22 carries out the functions described herein under the control of suitable software. This software may be downloaded to system 20 in electronic form, for example over a network. Alternatively or additionally, the software may be stored on tangible, non-transitory computer-readable media, such as optical, magnetic, or electronic memory media.

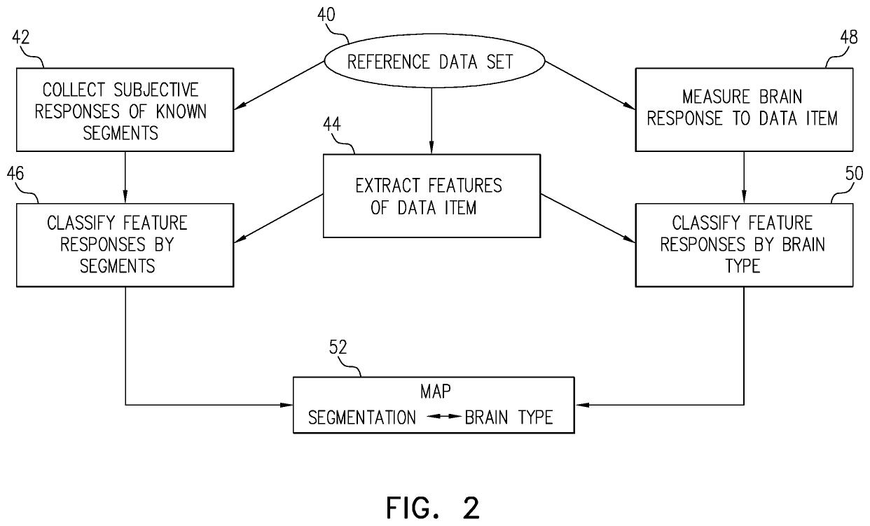

FIG. 2 is a flow chart that schematically illustrates a method for mapping between segmentation of an audience and brain types of the people in the audience, in accordance with an embodiment of the invention. As noted earlier, the segmentation may be in terms of demographic and/or psychographic information that is collected with respect to the reference group of subjects and used in defining mapping 28. The present method, as well as the details of implementation and application in the figures that follow, are described with reference to the components of system 20 for the sake of concreteness and clarity. Alternatively, the methods described herein may be implemented in other sorts of systems having suitable interfaces and computing and data storage capabilities. All such alternative implementations are considered to be within the scope of the present invention.

The method of FIG. 2 begins with a reference set 40 of data items, such as images. Processor 22 extracts features from the data items, at a feature extraction step 44. In the case of images, the extracted features may comprise spatial and/or spectral characteristics of the images. The features may be measured globally, for example in terms of the dominant colors, intensity, contrast, frequencies, and/or shapes in the images. Additionally or alternatively, processor 22 may extract local features from different parts of the images.

Alternatively or additionally, other sorts of data items may be used, and appropriate features of these data items may be extracted at step 44. For example:
The data items may comprise audio items, and the extracted features may include spectrograms and spectral characteristics of the audio waves.
The data items may comprise odors or flavors, and the extracted features may include spectroscopic data and chemical characteristics of the odors or flavors.
The data items may comprise tactile stimuli, and the extracted features may include vibrograms and spectral characteristics of the tactile stimuli.

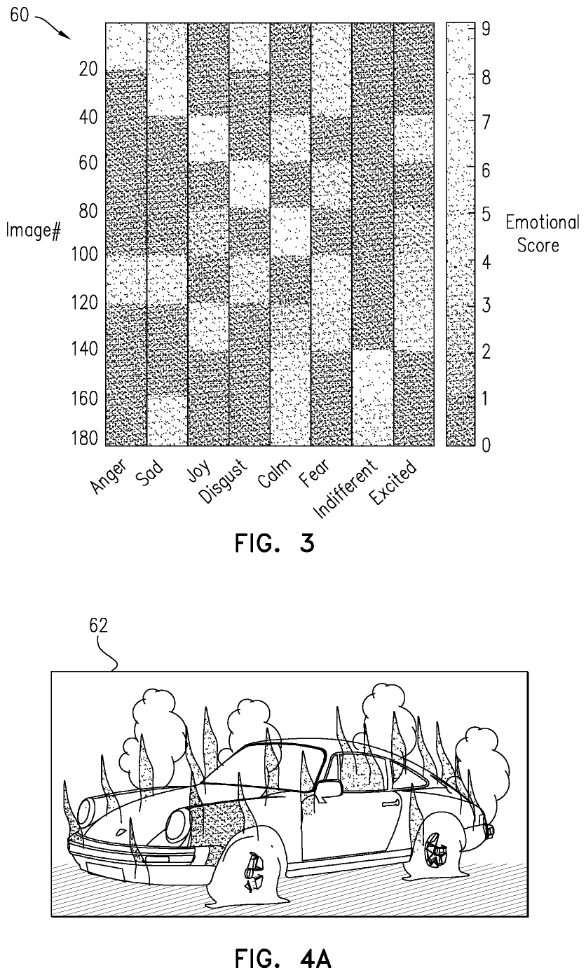

Processor 22 collects subjective responses to the images (or other data items) from the subjects in the reference group, at a subjective input step 42. The subjective responses may be collected, for example, by asking the subjects to rate the emotional impact of the images on a number of different scales, as illustrated in the figures that follow. Processor 22 correlates the subjective response scores with the image features extracted at step 44 and with the segmentation of the subjects and thus defines a classification of the subjective responses of the human subjects to each of the extracted features according to the segments to which the subjects belong, at a segment classification step 46. An example of this sort of classification is described below with reference to FIGS. 3-5.

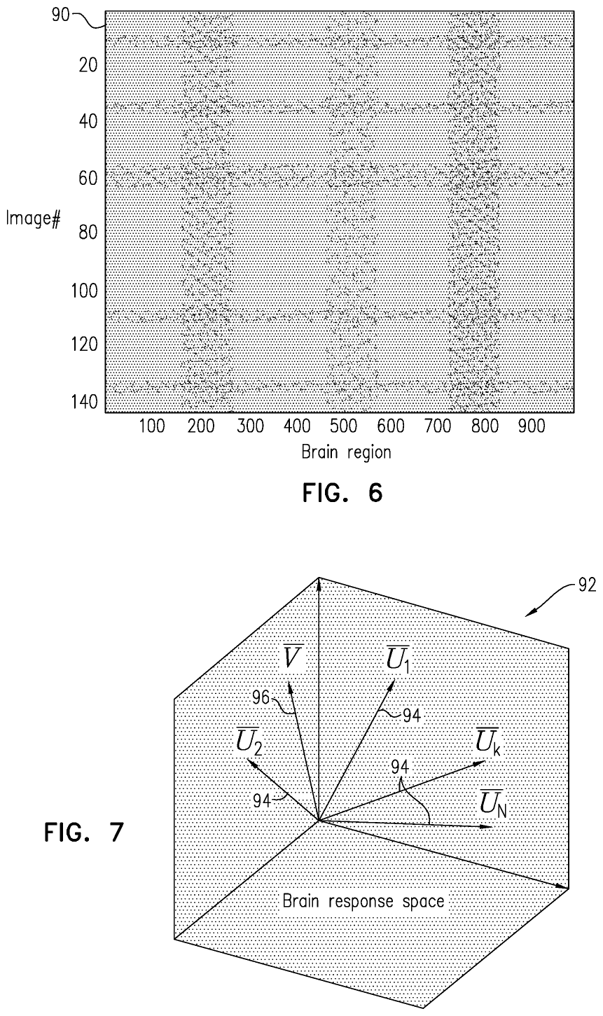

Processor 22 also receives the measured brain responses of the subjects in the reference group to each of the images (or other data items), at a brain response measurement step 48. As noted earlier, in the present embodiment these brain responses may comprise fMRI data, but other sorts of neurophysiological data may be used additionally or alternatively. Processor 22 parametrizes and clusters the brain responses, for example on the basis of the signals received from multiple different locations in the subjects' brains, to define a set of different brain types. The processor correlates the parametrized brain responses with the image features extracted at step 44 to define a classification of the neurophysiological responses of the human subjects to each of the extracted features according to the brain types of the subjects in the reference group, at a brain type classification step 50. This process is described further hereinbelow with reference to FIGS. 6 and 7.

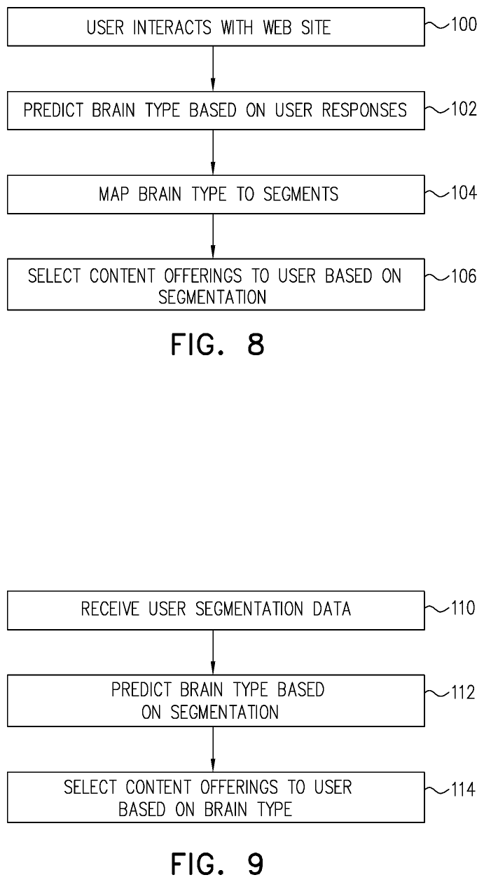

Processor 22 correlates the segment-based feature classification computed at step 46 with the brain type-based feature classification computed at step 50 in order to develop a mapping between the segmentation criteria and the brain types, at a mapping step 52. The mapping is bidirectional, i.e., it can be used in inferring segmentation information (including both demographic and psychographic segmentation) with respect to a given user 34 on the basis of the user's brain type, as indicated by responses to data items, for example images presented on a Web page; and conversely, it can be used in predicting the user's brain type based on segmentation information. These applications of the mapping derived at step 52 are described further hereinbelow with reference to FIGS. 8 and 9.

### Implementation Examples

FIG. 3 is a plot 60 that schematically illustrates emotional scoring of a set of images, in accordance with an embodiment of the invention. This sort of scoring is collected, for example, at step 42 in FIG. 2. The subjects view a large number of different images and, with respect to each image, rate their response in terms of certain specified emotions (anger, sadness, joy, etc.) on a scale of 0-9. Plot 60 illustrates the responses of one of the subjects.

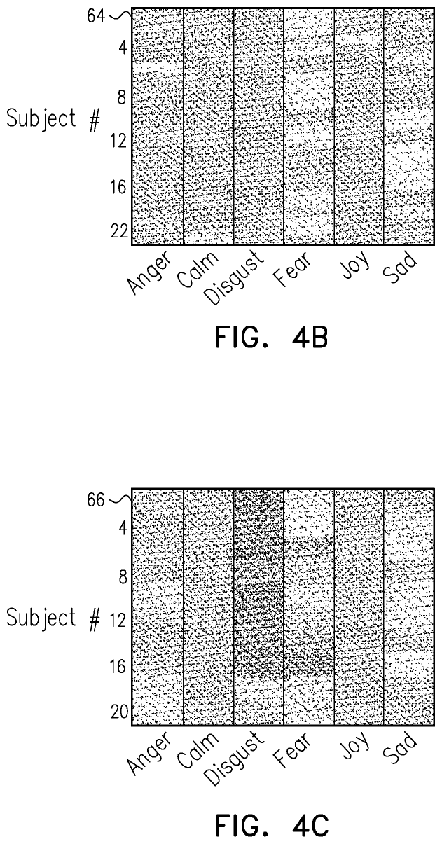

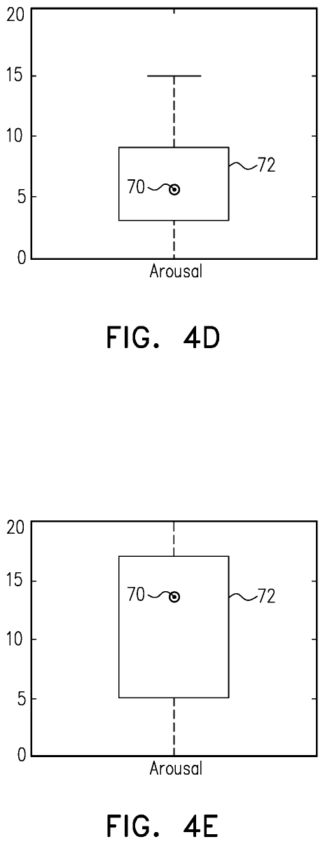

FIGS. 4A-E schematically illustrate an experiment in which subjective responses were collated and classified over two psychographic segments of subjects in a reference group, in accordance with an embodiment of the invention. One of the segments contained twenty subjects who classified themselves as politically liberal, while the other segment contained twenty subjects who classified themselves as politically conservative.

FIG. 4A shows a typical image 62 among the set of images that was used for this purpose, which included images intended to arouse strong emotions. FIGS. 4B and 4C are plots 64, 66 that schematically illustrate the aggregated emotional scoring of the image in FIG. 4A by the subjects in the two segments. Based on these aggregated subjective responses, an emotional arousal score for each segment was computed with respect to each of the images. FIGS. 4D and 4E are statistical plots showing the distribution of emotional arousal scores among the two segments, including median values 70 and ranges 72 between the 25% and 75% distribution limits in each segment. The differences between the distributions of arousal scores in the two segments were used in deriving a differential emotional arousal index with respect to each of the images.

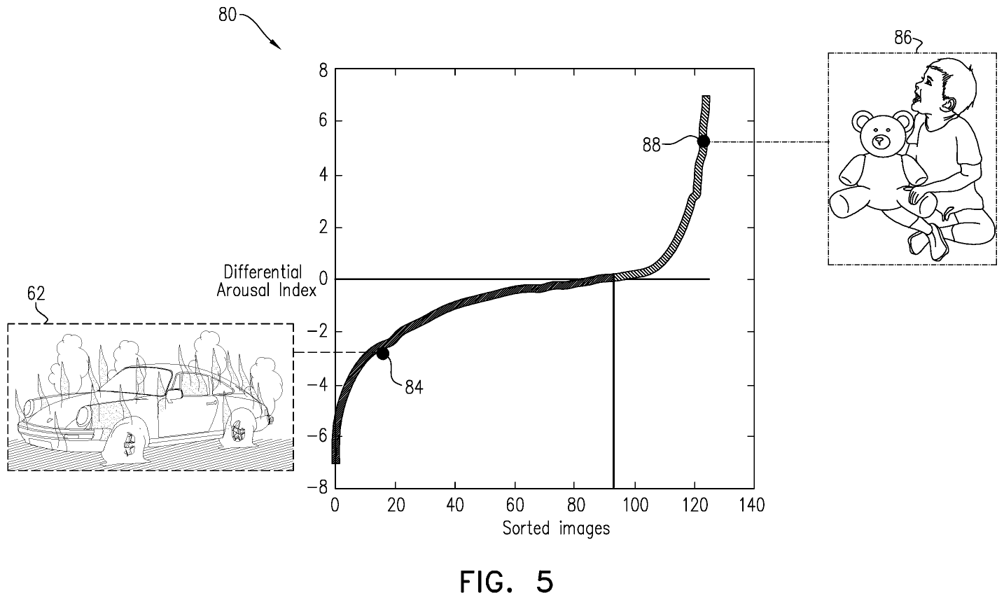

FIG. 5 is a plot 80 that schematically illustrates the range of values of the differential emotional arousal index computed between the two segments for each of the images in the experiment of FIGS. 4A-E, in accordance with an embodiment of the invention. The index in this example was computed by subtracting the average arousal score for each of the images among the subjects in the “conservative” segment from the corresponding average arousal score in the “liberal” segment. The images were sorted by the respective index values to generate plot 80. Images 62 and 86 in this example were found to be good differentiators between the segments, each with a substantially higher level of arousal in one segment than in the other.

Alternatively or additionally, processor 22 may extract features from each of the images, and then sort the features according to the arousal scores, weighted by the respective strengths of the features in each of the images. In this case, each of the features is classified according to the arousal score based on the subjective responses of the subjects to the images.

FIG. 6 is a plot 90 that schematically illustrates neurophysiological responses of a subject to a set of images, in accordance with an embodiment of the invention. In this example, the brain activity of the subjects in each of the segments was measured across multiple brain regions using fMRI while the subjects viewed each of the images in the set described above (or possibly only selected images that were found to be strong differentiators between the segments). The brain regions may be defined in terms of a grid of locations in the brain, for example, or in terms of anatomical structures within the brain. Alternatively, any suitable choice of signals may be collected from one or more regions of the subjects' brains for this purpose.

Processor 22 classified each of the images according to the measured brain activity in each region while the subjects viewed the images. Additionally or alternatively, image features may be classified in a similar fashion in terms of the brain activity that they arouse. For an image with features {yᵢ}, the brain activity can be expressed as a set of regional activity values {Rⱼ}, which are given by the formula Rⱼ=Σᵢβᵢⱼyᵢ, wherein βᵢⱼ represents the response of each region j to each feature i. Further details of the creation of brain activity maps and their use in data classification are described in the above-mentioned U.S. Patent Application Publications 2020/0170524 and 2021/0241065.

Alternatively or additionally, other types of measurements neurophysiological responses may be used by processor 22 in classifying the images and image features. For example, processor 22 may receive measurements of vital signs of the subjects (such as pulse and respiration rates, blood pressure, and skin conductivity) that were collected while the subjects viewed the images. As another alternative, processor 22 may process images or other recordings of the subjects to measure gestures made by the subjects while viewing the images.

FIG. 7 is a plot 92 that schematically illustrates a method of classifying brain types according to brain activity response, in accordance with an embodiment of the invention. Plot 92 is based on the respective signals collected (for example using fMRI as described above) from multiple regions of the brains of the subjects in the reference group. To classify the brain activity of these subjects, processor 22 clusters the respective signals to define a set of brain types, which are characterized by feature vectors 94 in a multi-dimensional brain response space, corresponding to respective average brain response vectors U₁, U₂, . . . U_{N}. Each of these brain types is also characterized by responses or actions performed by the corresponding subjects.

When a new subject, such as user 34, connects to system 20, processor 22 records a set of actions performed by the subject. For example, user 34 may be asked to select and/or rate images from the set viewed by the reference group. The responses of user 34 are recorded as actions {gₛₜ}sₜ, which are mapped to a vector 96 corresponding to an average brain response V in the brain response space of plot 92. The probability that user 34 has a brain type Ūₖ is then given by ∥ [symbol] V̅|Ūₖ [symbol] ∥².

Given a new subject with a predicted brain type Ξ̅ₖ, an inverse model can be used to estimate the probability that the new subject belongs to a demographic and/or psychographic segment represented by a vector Ψ̅ₖ in a segmentation space. Processor 22 calculates Ψ̅ₖ by computing a set of probabilities {∥ [symbol] Ξₖ|A⁻¹|Ūⱼ [symbol] ∥²}ⱼ, wherein A⁻¹ is a mapping between brain type space and subject segmentation space, and {Ūⱼ}ⱼ represents a set of brain types, as defined above. Statistical regression analysis may be used to find a mapping of brain type characteristics to a particular segmentation criterion, such as the subject's political views.

Thus, in the experiment described above, in which the reference group was divided into liberal and conservative segments, the average brain responses of the subjects in the reference group were measured for each of a number of the images that had high absolute values of the differential arousal index. Based on the average brain responses, processor 22 defined brain types corresponding to the subjects in each of the segments. New subjects, outside the reference group, were asked to rate the images, and processor 22 converted these ratings into a predicted brain type for each new subject, characterized by a brain response vector V. By finding the distance in the brain response space between V and the average brain types of the liberal and conservative segments, processor 22 was able to predict the political views of each new subject.

FIG. 8 is a flow chart that schematically illustrates a method for content selection based on mapping of brain types to segmentation of users of the content, in accordance with an embodiment of the invention. As in the example described above, this method can be used when an unknown person, such as user 34, interacts with a Web site, at a user interaction step 100. Processor 22 presents various data items, such as images, to user 34, and records the responses of user 34 to the presented data items. Based on these responses, processor 22 predicts the user's brain type, at a brain type prediction step 102. Alternatively or additionally, other sorts of user responses, such as the user's behavior, may be used in predicting the brain type.

Using mapping 28 between brain types and segments, which was derived from the reference group of subjects as described above, processor 22 maps the predicted brain type of user 34 to a demographic and/or psychographic segment, at a segment determination step 104. Processor 22 then selects content to offer to user 34 based on the segment to which the user has been mapped, at a content selection step 106.

FIG. 9 is a flow chart that schematically illustrates a method for content selection based on mapping of user segmentation to brain types, in accordance with an embodiment of the invention. In this example, processor 22 receives segmentation data, such as demographic and/or psychographic data, with respect to a new subject, such as user 34, at a segmentation input step 110. The segmentation data may be input voluntarily by user 34, for example, or it may be culled from other sources of information. Based on the segmentation data, processor 22 uses mapping 28 to predict the brain type of user 34. System 20 may then select content to present to user 34 that is suited to the user's brain type, at a content selection step 114.

For example, assuming system 20 has stored a menu of products with feature vectors {hₐ}_{a=1 to A} or clusters of products with representative feature vectors {q_{b}}_{b=1 to b}, which respectively match predicted brain responses {Hₐ}_{a=1 to A} and {Q_{b}}_{b=1 to b}, the probabilities that the user will be interested in these products is given by {∥ [symbol] Hₐ [symbol] ∥²}_{a=1 to A} and {∥ [symbol] Q_{b} [symbol] ∥²}_{b=1 to B}.

Furthermore, the predicted brain response may include features relating to the state of mind, mood, and/or intent of user 34. System 20 may store another set of feature vectors {λₛₜ}ₛ_{ₜ} that corresponds to the user's current state of mind, mood, and/or intent. In this case, the content offered at step 114 may change to fit the user's current state, according to the calculated probabilities.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for content delivery, comprising:
dividing a reference group of human subjects into multiple segments according to one or more segmentation criteria;
collecting subjective responses of the human subjects to a reference set of data items;
measuring neurophysiological responses of the human subjects to the data items in the reference set;
classifying the human subjects into multiple brain types according to the measured neurophysiological responses;
based on the collected subjective responses, defining a mapping between the segmentation criteria and the brain types;
applying the mapping in predicting a brain type of a human subject outside the reference group; and
selecting a content offering for presentation to the human subject responsively to the predicted brain type.

2. The method according to claim 1, wherein applying the mapping comprises predicting the brain type based on a behavior of the human subject.

3. The method according to claim 1, wherein applying the mapping comprises predicting the brain type based on an interaction of the human subject with an item of content.

4. The method according to claim 1, wherein the segmentation criteria comprise demographic criteria.

5. The method according to claim 1, wherein the segmentation criteria comprise psychographic criteria.

6. The method according to claim 5, wherein the psychographic criteria comprise one or more measures of mental health of the human subjects.

7. The method according to claim 1, wherein selecting the content offering comprises presenting a media item to the human subject.

8. The method according to claim 1, wherein selecting the content offering comprises modifying a physical property of an output presented to the human subject.

9. The method according to claim 1, wherein selecting the content offering comprises presenting a proposal to the human subject to make an acquaintance with another person.

10. The method according to claim 1, wherein selecting the content offering comprises presenting a proposal to the human subject to join an organization.

11. The method according to claim 1, wherein measuring the neurophysiological responses comprises collecting respective signals from one or more region of respective brains of the human subjects, and wherein classifying the human subjects comprises clustering the human subjects according to the respective signals.

12. The method according to claim 11, wherein collecting the respective signals comprises receiving magnetic resonance imaging (MRI) data.

13. The method according to claim 1, wherein measuring the neurophysiological responses comprises sensing vital signs of the human subjects.

14. The method according to claim 1, wherein measuring the neurophysiological responses comprises sensing gestures made by the human subjects.

15. The method according to claim 1, wherein measuring the neurophysiological responses comprises measuring a dilation of pupils of the eyes of the human subjects.

16. The method according to claim 1, wherein defining the mapping comprises:
extracting features from the data items;
defining a first classification of the neurophysiological responses of the human subjects to each of the extracted features according to the brain types of the human subjects;
defining a second classification of the subjective responses of the human subjects to each of the extracted features according to the segments to which the human subjects belong; and
applying the first and second classifications in mapping between the segmentation criteria and the brain types.

17. The method according to claim 16, wherein the data items comprise images, and the extracted features are selected from among spatial and spectral characteristics of the images.

18. The method according to claim 16, wherein the data items comprise audio items, and the extracted features are selected from among spectrograms and spectral characteristics of the audio waves.

19. The method according to claim 16, wherein the data items comprise odors, and the extracted features are selected from among spectroscopic data and chemical characteristics of the odors.

20. The method according to claim 16, wherein the data items comprise flavors, and the extracted features are selected from among spectroscopic data and chemical characteristics of the flavors.

21. The method according to claim 16, wherein the data items comprise tactile stimuli, and the extracted features are selected from among vibrograms and spectral characteristics of the tactile stimuli.

22. The method according to claim 16, wherein defining the first classification comprises measuring a brain activity of the human subjects from one or more brain regions, and classifying each of the features according to the measured brain activity.

23. The method according to claim 16, wherein defining the second classification comprises computing an arousal score with respect to each of the data items based on the subjective responses, and classifying each of the features according to the arousal score.

24. The method according to claim 1, wherein predicting the brain type comprises presenting a data item to the human subject, receiving a response of the human subject to the presented data item, and predicting the brain type based on the received response.

25. The method according to claim 1, wherein predicting the brain type comprises receiving segmentation data with respect to the human subject, and predicting the brain type based on the segmentation data.

26. Apparatus for content delivery, comprising:
a memory, configured to receive and store subjective responses of a reference group of human subjects to a reference set of data items and to receive and store neurophysiological responses of the human subjects to the data items in the reference set; and
a processor, configuring to divide the reference group of human subjects into multiple segments according to one or more segmentation criteria, to classify the human subjects into multiple brain types according to the stored neurophysiological responses, to define, based on the stored subjective responses, a mapping between the segmentation criteria and the brain types, to apply the mapping in predicting a brain type of a human subject outside the reference group, and to select a content offering for presentation to the human subject responsively to the predicted brain type.

27. The apparatus according to claim 26, wherein the processor is configured to predict the brain type based on a behavior of the human subject.

28. The apparatus according to claim 26, wherein the processor is configured to predict the brain type based on an interaction of the human subject with an item of content.

29. The apparatus according to claim 26, wherein the segmentation criteria comprise demographic criteria.

30. The apparatus according to claim 26, wherein the segmentation criteria comprise psychographic criteria.

31. The apparatus according to claim 30, wherein the psychographic criteria comprise one or more measures of mental health of the human subjects.

32. The apparatus according to claim 26, wherein the selected content offering comprises a media item presented to the human subject.

33. The apparatus according to claim 26, wherein the selected content offering comprises a modification of a physical property of an output presented to the human subject.

34. The apparatus according to claim 26, wherein the selected content offering comprises a proposal presented to the human subject to make an acquaintance with another person.

35. The apparatus according to claim 26, wherein the selected content offering comprises a proposal presented to the human subject to join an organization.

36. The apparatus according to claim 26, wherein the neurophysiological responses are measured by collecting respective signals from one or more regions of respective brains of the human subjects, and wherein the processor is configured to classify the human subjects by clustering the human subjects according to the respective signals.

37. The apparatus according to claim 36, wherein the collected signals comprise magnetic resonance imaging (MRI) data.

38. The apparatus according to claim 26, wherein the neurophysiological responses are measured by sensing vital signs of the human subjects.

39. The apparatus according to claim 26, wherein the neurophysiological responses are measured by sensing gestures made by the human subjects.

40. The apparatus according to claim 26, wherein the neurophysiological responses are measured by measuring a dilation of pupils of the eyes of the human subjects.

41. The apparatus according to claim 26, wherein the processor is configured to define the mapping by extracting features from the data items, defining a first classification of the neurophysiological responses of the human subjects to each of the extracted features according to the brain types of the human subjects, defining a second classification of the subjective responses of the human subjects to each of the extracted features according to the segments to which the human subjects belong, and applying the first and second classifications in mapping between the segmentation criteria and the brain types.

42. The apparatus according to claim 41, wherein the data items comprise images, and the extracted features are selected from among spatial and spectral characteristics of the images.

43. The apparatus according to claim 41, wherein the data items comprise audio items, and the extracted features are selected from among spectrograms and spectral characteristics of the audio waves.

44. The apparatus according to claim 41, wherein the data items comprise odors, and the extracted features are selected from among spectroscopic data and chemical characteristics of the odors.

45. The apparatus according to claim 41, wherein the data items comprise flavors, and the extracted features are selected from among spectroscopic data and chemical characteristics of the flavors.

46. The apparatus according to claim 41, wherein the data items comprise tactile stimuli, and the extracted features are selected from among vibrograms and spectral characteristics of the tactile stimuli.

47. The apparatus according to claim 41, wherein the first classification is defined by measuring a brain activity of the human subjects from one or more brain regions, and classifying each of the features according to the measured brain activity.

48. The apparatus according to claim 41, wherein the second classification is defined by computing an arousal score with respect to each of the data items based on the subjective responses, and classifying each of the features according to the arousal score.

49. The apparatus according to claim 26, wherein the processor is configured to present a data item to the human subject, to receive a response of the human subject to the presented data item, and to predict the brain type based on the received response.

50. The apparatus according to claim 26, wherein the processor is configured to receive segmentation data with respect to the human subject, and to predict the brain type based on the segmentation data.

51. A computer software product, comprising a tangible, non-transitory computer-readable medium in which program instructions are stored, which instructions, when read by a computer, cause the computer to receive and store subjective responses of a reference group of human subjects to a reference set of data items and to receive and store neurophysiological responses of the human subjects to the data items in the reference set, and to divide the reference group of human subjects into multiple segments according to one or more segmentation criteria, to classify the human subjects into multiple brain types according to the stored neurophysiological responses, to define, based on the stored subjective responses, a mapping between the segmentation criteria and the brain types, to apply the mapping in predicting a brain type of a human subject outside the reference group, and to select a content offering for presentation to the human subject responsively to the predicted brain type.
